(19) United States
(12) Patent Application Publication
(10) Pub. No.: US 2023/0023285 A1
(43) Pub. Date: Jan. 26, 2023

(54) PHARMACEUTICAL COMPOSITION FOR USE IN PREVENTING OR STOPPING METASTASES

(71) Applicant: LOGICK ENERGETICS B.V., Leiden (NL)

(72) Inventors: Gerard KRIELEN, Leiden (NL); Clemens Waltherus Gerardus Maria LÖWIK, Leiden (NL)

(73) Assignee: LOGICK ENERGETICS B.V., Leiden (NL)

(21) Appl. No.: 17/786,104

(22) PCT Filed: Dec. 16, 2020
(86) PCT No.: PCT/NL2020/050795

§ 371 Date: Jun. 16, 2022

(30) Foreign Application Priority Data
Dec. 18, 2019 (NL) 2024493

(51) Int. Cl. A61K 31/737; A61P 35/00; A61K 31/405; A61K 31/4439

(52) U.S. Cl. CPC A61K 31/737; A61P 35/00; A61K 31/405; A61K 31/4439

(57) ABSTRACT

The invention relates to a composition, and a medicament for preventing or stopping of metastases, in particular metastases originating from epithelia cancer, such as skin cancer, lung cancer, stomach cancer, brain cancer, breast cancer, colo-rectal cancer, prostate cancer, pancreas cancer, head/neck cancer, and esophagus cancer. It has been found that the present composition irreversibly converts cancer cells with metastatic potential into harmless cells with no metastatic potential.

### FIELD OF THE INVENTION

The invention relates to a composition, and a medicament for the prevention of metastasis or stopping of ongoing metastasis, in particular metastases originating from epithelial cancers, such as skin cancer, lung cancer, breast cancer, colorectal cancer, prostate cancer, liver cancer, stomach cancer, brain cancer, kidney cancer, gall bladder cancer, pancreas cancer, head/neck cancer, bladder cancer, melanomas, and esophagus cancer. It has been found that the present composition prevents or stops metastasis.

### BACKGROUND OF THE INVENTION

The occurrence of cancers is widely spread. For treatment of these cancers typically aggressive drugs are used, such as chemotherapy, which pose many typically negative side-effects to the human body. The treatment of metastases is even worse, as these metastases are often spread throughout the body, and can therefore typically not be treated effectively, at least not without causing severe damage to the body.

Cancer relates to cells that proliferate in a largely uncontrolled manner, and therefore compared to other living cells they proliferate more rapid and also endlessly. Therewith a primary tumour is formed. In general metastasis relates to a spread from an initial cancer site to a different site within a living body. These different sites may then be referred to as metastases. This spread is caused by cancer cells which are able to circulate through the body towards other parts thereof. There they may form a secondary tumour, the metastatic tumour. The metastasis may have a small volume, but it is often widely spread through the body. Most cancers can metastasize, of which some are in particular troublesome. The cells of the metastasis often resemble the cells of the original tumour, and therefore reference is made to the initial tumour when characterizing the metastasis. They could therefore in principle be treated likewise, but as they are widespread, treatment may be cumbersome and to harmful to the body. However, metastases can acquire new characteristics making them unresponsive to anti-cancer drugs that are effective against the primary tumour, making them even more difficult to treat. Currently, primary tumours that have not metastasized can effectively be treated by surgery, usually in combination with chemotherapy and/or radiation-therapy. This is the major reason why cancer patients often do not die from their primary tumour but from the metastases. Therefore, there is an unmet need for new treatments that can prevent metastasis or stop ongoing metastasis.

For treatment of cancer metastases often combined therapies are used, that is two or more chemicals are combined. These combined therapies are often more effective. Still, typically for every individual human being such as therapy needs to be established, which often involves testing of therapies that after evaluation thereof are considered to be ineffective.

Some documents may be referred to. WO 88/05301 recites sulphated polysaccharides as such, having anti-metastatic and/or anti-inflammatory activity, a method of anti-metastatic and/or anti-inflammatory treatment of an animal or human patient comprising administration to the patient of an effective amount of at least one sulphated polysaccharide which blocks or inhibits endoglycosidase, particularly heparanase, activity. Suitable sulphated polysaccharides include heparin and modified heparin, fucoidan, pentosan sulphate, dextran sulphate and carrageenan lambda. WO 96/33726 A1 recites preparation and use of sulphated oligosaccharides, wherein the oligosaccharide has the general formula (I): R₁—(Rₓ)ₙ—R₂, wherein R₁ and R₂ and each Rₓ represents a monosaccharide unit, all of which may be the same or different, adjacent monosaccharide units being linked by 2, 3, 4 and/or 6 glycosidic bonds and n is an integer from 1 to 6, and use thereof as anti-angiogenic, anti-metastatic and/or anti-inflammatory agents. WO 2011/113892 A2 recites a combined use of phospholipid and sulphate groups-carrying polysaccharides for inhibiting metastatic spread, and to a medicament comprising a phospholipid component and a polysaccharide component having polysaccharides containing sulphate groups for inhibiting metastatic spread of tumours. The two components may be in same or separate preparation. Panigraphy et al. in “PPARγ as a therapeutic target of tumour angiogenesis and metastasis” in Cancer Biology&Therapy 4:7, 687-693, July 2005, review the action of PPARγ agonists on angiogenesis and inflammation in the context of tumorigenesis as an integrated tissue process. They discuss potential explanations for the conflicting effects of PPARγ agonists on tumour progression and metastasis. Sorting out the various modes of action and defining their relative contribution in the context of tumour and host tissue as a heterogeneous target will therefore be crucial to understand the multi-facetted effects of PPARγ. WO 2018/132899 A1 recites a method for treating, preventing, reducing, or delaying onset of, osteosarcoma lung metastasis in a subject, comprising: administering a therapeutically or prophylactically effective amount of a PPARγ agonist, an anti-inflammatory agent, or a neutrophil depleting agent, to a subject having or suspected of having osteosarcoma. WO 2017/105229 A1 recites a pharmaceutical composition for inducing beige or brown fat tissue, comprising (i) a compound comprising at least one carbohydrate and containing at least one sulphate, in combination with (ii) at least one non-protein or non-NA-strand compound, different from (i), that can activate PPAR, preferably PPARγ, and (iii) optionally, at least one pharmaceutically acceptable carrier. Preferably, the sulphated compounds include single sulphur containing agents like MSM or multiple sulphur containing agents like pentosan, adequan. The second part of the composition, is a PPARγ agonists chosen from the group of thiazolidinediones, NSAIDs, sulphonyl ureas and indoles, like for example indomethacin or pioglitazone.

As therapies for treatment of metastases are scarce or ineffective, there still is a need for improved pharmaceuticals and treatments which overcome drawbacks of the prior art.

### SUMMARY OF THE INVENTION

It is an objective of the invention to provide a composition that is able to prevent metastasis or stop metastasis, such as stopping ongoing metastasis or stopping further metastasis, by irreversibly blocking the metastatic capacity of tumor cells. The invention furthermore provides method of use of such composition, and for the prevention of metastasis and stopping of ongoing metastasis, especially if a mammal is prone to develop metastases, such as in cancer phase II or III, and for limiting or containing effects thereof. The present invention provides in fact two synergistic aspects of the first compound comprising at least one carbohydrate and containing more than one sulphate and the second compound selected from the group of thiazolidinediones, NSAIDs, sulphonylureas, and indoles, namely (A) (A1) stopping of ongoing metastasis in addition to (A2) prevention of metastasis, and (B) the combination of first compound and second compound gives, compared to the expression of the sum of both compounds applied separately, an at least two-fold expression of certain genes (e.g. FABP4, CD36, PLIN1). For effect (A1) the breast-cancer cell line shows differentiation (change in cell type) of these cells into brown/beige and likewise white fat cells. As a consequence, ongoing metastasis is stopped. For effect (A2) a cancer cell is found to differentiate towards a stem cell which on its turn is differentiated into a brown/beige fat cell, under influence of the two above compounds. Two mouse cell-lines as well as cancer cell-lines are considered to show this effect. In the cancer cell-lines virtually “no” new cancer cells are formed, but rather the fat cells. Hence metastasis is prevented. For effect (B) the experimental results and added results give clear evidence.

The invention provides for a pharmaceutical composition for the prevention of metastasis and stopping of ongoing metastasis, comprising a first compound comprising at least one carbohydrate and containing more than one sulphate, and a second compound, different from the first compound, that can activate PPAR, preferably PPARγ. The first compound preferably comprises two or more sulphates, and is more preferably an oligo-, or polysulphate comprising compound, such as comprising 3-20 sulphates, preferably containing 4-15 sulphates, more preferably containing 5-12 sulphates, such as 6-10 sulphates, e.g. 7-8 sulphates, such as (C₅H₆XᵢO10S2)ₙ, wherein X may be selected from single valence metal atoms, such as Na⁺, and K⁺, i being 2, and from double valence metal atoms, such as Ca²⁺, and Mg²⁺, i being 1, and n preferably being in the range of 2-20, more preferably 3-12, such as 4-8. When reference to the first compound is made the references includes the oligo- and polysulphates, as indicated above. The molecular weight of the first compound is preferably not too large, such as <10 kDa, preferably <7 kDa, such as <5 kDa. A compound that can activate PPAR means that the PPAR receptor activity increases by at 2 to 3 times compared to a baseline situation without the PPAR activating compound. The compound that can activate PPAR is not a protein, or not a nucleic acid (NA)-strand compound. Suitable compounds that can activate PPAR include thiazolidinediones, NSAIDs, sulphonylureas and indoles. Inventors discovered that metastasis can be prevented or ongoing metastasis can be stopped by a composition according to the invention. The composition comprises a carbohydrate comprising more than one sulphate group like Pentosan polysulphate in combination with a PPAR, preferably a PPARγ agonist, like indomethacin or pioglitazone stimulated synergistically the formation of adipocytes with Beige/Brite and/or BAT characteristics, that is an irreversible transfer of cancer cells with metastatic potential into harmless cells. It is noted that white adipocyte, beige adipocyte, and brown adipocyte clearly distinguish from one and another, such as being UCP1 negative or positive, size of lipid droplets, adipokine secretion, and so on. Hence formation of beige/brite cells is not too difficult to establish. Such a transfer can be observed in the mesenchymal progenitor KS483 cell line, deposited at DSMZ, Inhoffenstr. 7B, D-38124 Braunschweig, Germany, under accession number DSM ACC3286 on Jan. 13, 2016, but other cell lines of comparable origin can be used as well, such as the 3T3-L1 cell line which may be obtained from Sigma-Aldrich, and also epithelial cancer cell lines with metastatic potential.

The invention therefore provides a pharmaceutical composition, comprising a compound comprising at least one carbohydrate (saccharide) and containing at least one sulphate, in combination with a second compound that can activate PPAR, preferably PPARγ, for use in the prevention of metastasis or stopping of ongoing metastasis.

Hence, patients with cancers that are known to metastasize or that already have one or more metastases, benefit from use of the pharmaceutical composition of the invention because metastasis can be prevented or ongoing metastasis can be stopped.

Therefore, the invention also relates to the use of the pharmaceutical composition as described as a medicament.

Preferably, the invention further relates to the pharmaceutical composition of the invention, for use in the prevention of metastasis or stopping of ongoing metastasis of these metastatic cancers.

### DETAILED DESCRIPTION OF THE INVENTION

In an exemplary embodiment the present pharmaceutical composition for use in the prevention of metastasis or stopping of ongoing metastasis may comprise pentosan sulphate, and/or adequan, Adequan being a PolySulfated GlycosAminoGlycan (PSGAG).

In an exemplary embodiment the present pharmaceutical composition for use in the prevention of metastasis or stopping of ongoing metastasis may comprise indomethacin and/or pioglitazone.

In an exemplary embodiment the present pharmaceutical composition for use in the prevention of metastasis or stopping of ongoing metastasis may comprise pentosan sulphate and/or adequan, and indomethacin and/or pioglitazone.

In an exemplary embodiment the present pharmaceutical composition for use in the prevention of metastasis or stopping of ongoing metastasis may comprise pentosan sulphate and indomethacin, pentosan sulphate and pioglitazone, adequan and indomethacin, adequan and pioglitazone, as well as combinations thereof.

In an exemplary embodiment of the present pharmaceutical composition for use in the prevention of metastasis or stopping of ongoing metastasis the first compound and second compound may be provided in a molar ratio of 0.01:1 to 1:0.01, preferably 0.2:1 to 1:0.2, more preferably 0.33:1 to 1:0.33, even more preferably 0.45:1 to 1:0.245, such as 0.5:1 to 1:0.5, e.g. 0.9:1 to 1:0.9.

In an exemplary embodiment of the present pharmaceutical composition for use in the prevention of metastasis or stopping of ongoing metastasis the composition further comprises (iii) at least one pharmaceutically acceptable carrier. In an exemplary embodiment of the present pharmaceutical composition for use in the prevention of metastasis or stopping of ongoing metastasis the active pharmaceutical ingredients are in one dosage form, preferably comprising 1-10 mg active ingredients/kg body weight, preferably 2-5 mg active ingredients/kg body weight, such as 2-1000 mg active ingredients, preferably 10-500 mg active ingredients, more preferably 20-100 mg active ingredients.

In an exemplary embodiment of the present pharmaceutical composition for use in the prevention of metastasis or stopping of ongoing metastasis the pharmaceutical composition may comprise separate dosage forms for individual pharmaceutical active ingredients, preferably 2-10 dosage forms, such as 3-5 dosage forms.

In an exemplary embodiment of the present pharmaceutical composition for use in the prevention of metastasis or stopping of ongoing metastasis the composition may be in the form of a tablet, capsule, repository, or injectable.

In an exemplary embodiment of the present pharmaceutical composition for use in the prevention of metastasis or stopping of ongoing metastasis the composition may be in the form of a tablet or capsule suitable for oral administration.

In an exemplary embodiment the present pharmaceutical composition for use in the prevention of metastasis or stopping of ongoing metastasis may be formulated in a form, which, depending on its use, allows targeting of specific cells, tissues or organs, such as in the form of nanoparticles. For example, the nanoparticle may be any kind of nanodevice in which both compounds are packaged for targeted delivery. In a more preferred embodiment, targeting can be done by any specific molecule attached on the outside of the specific form, such as for example a nanoparticle, like an antibody, or other molecular or chemical entity that can target specific cells, tissues or organs.

In an exemplary embodiment the present pharmaceutical composition for use in the prevention of metastasis or stopping of ongoing metastasis the dosage and dosage form are such that all metastases in a body are targeted.

In an exemplary embodiment of the present pharmaceutical composition for use in the prevention of metastasis or stopping of ongoing metastasis the cancer metastasis may originate from epithelial cancers.

In an exemplary embodiment of the present pharmaceutical composition for use in the prevention of metastasis or stopping of ongoing metastasis the cancer may be selected from skin cancer, stomach cancer, brain cancer, lung cancer, breast cancer, colorectal cancer, liver cancer, kidney cancer, gall bladder cancer, prostate cancer, bladder cancer, pancreas cancer, head/neck cancer, and esophagus cancer.

In an exemplary embodiment the present pharmaceutical composition for use in the prevention of metastasis or stopping of ongoing metastasis may be for use as a medicament by administering said medicament in an effective amount to epithelial cancers for a sufficient period. Typically, the present pharmaceutical composition may be used over a period of 1 week-12 months, preferably 2 weeks-6 months, such as 4 weeks-3 months. Thereafter the pharmaceutical composition may be used for an additional period of time, typically in a lower dosage, such as 10-50% of an initial dosage, wherein the additional period may be at least one month, and typically it may be used for more than a year, such as a five to ten years or even during a remainder of the life time of the pet or mammal.

In an exemplary embodiment of the present pharmaceutical composition for use in the prevention of metastasis or stopping of ongoing metastasis the administration may be to a pet or mammal comprising said tissue, wherein the mammal preferably is a human with metastasis.

In an exemplary embodiment of the present pharmaceutical composition for use in the prevention of metastasis or stopping of ongoing metastasis may be as a medicament.

More in particular, the invention also relates to pharmaceutical composition of the invention for use in the prevention of metastasis or stopping of ongoing metastasis.

The pharmaceutical composition according the invention may be a combination composition, wherein the active pharmaceutical ingredients are in one dosage form. The pharmaceutical composition may also comprise separate dosage forms for individual pharmaceutical active ingredients.

The compound comprising at least one carbohydrate and containing at least one sulphate preferably has anti-inflammatory effects, meaning that at least one of the inflammatory markers is reduced. Inflammatory markers are e.g. TNFα, IFN, cytokines, histamine, interleukins, chemokines, leukotrienes, lysosome granules and prostaglandins.

The compound that activates PPAR, preferably PPARγ preferably is a compound that can activate PPAR, meaning the PPAR receptor activity increases by at least 2-3 times compared to a baseline situation without the PPAR activating compound.

The compound that activates PPAR, preferably PPARγ preferably has anti-inflammatory effects, meaning that at least one of the inflammatory markers is reduced. Inflammatory markers are e.g. TNFα, IFN, cytokines, histamine, interleukins, chemokines, leukotrienes, lysosome granules and prostaglandins.

A carbohydrate is an organic compound comprising only carbon, hydrogen, and oxygen, usually with a hydrogen:oxygen atom ratio of 2:1 (as in water); with the empirical formula Cₘ(H₂O)ₙ (where m could be different from n). Structurally it is more accurate to view carbohydrates as polyhydroxy aldehydes and ketones.

Carbohydrates as used in the present invention do not relate to glycosylated proteins or nucleoside compounds (like DNA, RNA or the like)

The carbohydrate preferably is a Glycosaminoglycans (GAGs) or keratan. Glycosaminoglycans, or mucopolysaccharides are long unbranched polysaccharides consisting of a repeating disaccharide unit. The repeating unit consists of an amino sugar (N-acetylglucosamine or N-acetyl galactosamine) along with a uronic sugar (glucuronic acid or iduronic acid) or galactose.

Based on core disaccharide structures, GAGs may be classified into four groups.

Preferred types of sulphated saccharides are also known as heparin, heparan sulphate analogues, or heparin-like compounds.

Suitable compounds comprising a carbohydrate and a sulphate group can be derived from natural sources, or can be made—at least in part—synthetically.

Examples of sulphated compounds include single Sulphur containing agents like MSM (dimethylsulfon), dextran sulphate, or multiple Sulphur containing agents, like polysulphated glycosaminoglycan (adequan), heparin like pentosan sulphate or the like.

Preferred sulphated saccharides compounds are polysulphated glycosaminoglycan (Adequan), heparin like pentosan polysulphate (Elmiron) or the like.

The second part of the present composition, is an agent that can activate PPAR, preferably PPARγ. Preferred PPAR agonists include triglitazones (TZDs: triglitazone, rosiglitazone, pioglitazone etc.) and indomethacin.

Preferably, both the compound comprising at least one carbohydrate and containing at least one sulphate and the compound that activates PPARγ preferably has anti-inflammatory effects, meaning that at least one of the inflammatory markers is reduced. Inflammatory markers are e.g. TNFα, IFN, cytokines, histamine, interleukins, chemokines, leukotriene, lysosome granules and prostaglandins.

Whether a compound is a PPAR, preferably PPARγ agonist can be determined in a simple cell-based test.

By PPAR agonist is meant any compound that increases the biological activity or expression of one or more PPARs (e.g., PPARα, PPARγ, and PPARβ/δ) in a cell by a least 10% relative.

Examples of PPARα agonists include any of the Thiazolidinediones, but particularly Rosiglitazone, Troglitazone and Pioglitazone and analogs thereof. Rosiglitazone and pioglitazone are the preferred PPAR agonists. Additional examples of PPARγ agonists include non-steroidal anti-inflammatory drugs, such as Indomethacin, Ibuprofen, Naprosyn and Fenoprofen and antioxidants such as vitamin E, vitamin C, S-adenosyl methionine, selenium, beta-carotene, idebenone, cysteine, dithioerythritol, dithionite, dithiothreitol, and pyrosulfite.

Examples of PPARα agonists include any of the fibrates (e.g., fenofibrate, bezafibrate, gemfibrozil, and analogs thereof), docosahexaenoic acid, and Wy 4643.

So, for example Pentosan sulphate in combination with one of these compounds could prevent metastasis or stop ongoing metastasis. More specifically PPARγ agonistic action can be determined in a similar manner as described above, using a dual luciferase assay with a luciferase construct containing a PPARγ responsive element.

This second compound is not a protein or nucleic acid-based compound.

Suitable PPARγ agonists include thiazolidinediones, NSAIDs, sulphonyl ureas and indoles.

Thiazolidinediones (abbreviated as TZDs) are also known as glitazones. Suitable thiazolidinediones include rosiglitazone, pioglitazone, troglitazone, and ciglitazone, which are selective ligands for the nuclear transcription factor peroxisome proliferator-activated receptor (PPAR)y. Typical TZDs have the formula as shown in FIG. 5, where n is 1, 2, or 3, Y and Z independently are O or NH; and E is a cyclic or bicyclic aromatic or non-aromatic ring, optionally containing a heteroatom selected from oxygen or nitrogen. Suitable TZDs are for example described in WO2000/27401.

NSAIDs are a class of drugs that provides analgesic and antipyretic (fever-reducing) effects, and, in higher doses, anti-inflammatory effects. PPARγ is activated by several endogenous ligands emerging from the metabolism of arachidonic acid and linoleic acid. Among the PPARγ ligands represented are the lipoxygenase products 13(S)HODE (produced from linoleic acid by 15-LOX-1) and 15(S)HETE (produced from arachidonic acid by both 15-LOX-1 and 15-LOX-2, although 15-LOX-2 catalyzes this reaction much more efficiently). Induction of 15-LOX-1 activity by NSAIDs occurs independently of COX-2 inhibition

NSAIDs can be classified based on their chemical structure or mechanism of action. Older NSAIDs were known long before their mechanism of action was elucidated and were for this reason classified by chemical structure or origin. Newer substances are more often classified by mechanism of action.

Suitable NSAIDs include salicylates, propionic acid derivatives, acetic acid derivatives, enolic acid (oxicam) derivatives and fenamic acid derivatives,

Suitable salicylates include aspirin (acetylsalicylic acid), diflunisal (Dolobid™), salsalate (Disalcid™) and choline magnesium trisalicylate (Trilisate™)

Suitable propionic acid derivatives include ibuprofen, dexibuprofen, naproxen, fenoprofen, ketoprofen, dexketoprofen, flurbiprofen, oxaprozin, and loxoprofen.

Suitable acetic acid derivatives include Indomethacin, tolmetin, sulindac, etodolac, ketorolac, diclofenac and nabumetone (drug itself is non-acidic but the active, principal metabolite has a carboxylic acid group)

Suitable enolic acid (oxicam) derivatives include piroxicam, meloxicam, tenoxicam, droxicam, lornoxicam, and isoxicam.

Suitable fenamic acid derivatives (fenamates) include mefenamic acid, meclofenamic acid, flufenamic acid, and tolfenamic acid.

Sulfonylurea derivatives are a class of antidiabetic drugs that are used in the management of diabetes mellitus type 2. Examples include carbutamide, acetohexamide, chlorpropamide, tolbutamide, tolazamide, glipizide, gliclazide, glibenclamide (glyburide), glibornuride, gliquidone, glisoxepide, glyclopyramide and glimepiride. They act primarily by increasing insulin release from the beta cells in the pancreas. All sulfonylureas contain a central S-phenyl sulfonylurea structure with a p-substituent on the phenyl ring (R) and various groups terminating the urea N′ end group (R2) (see FIG. 6).

Indoles include indole-derived agents which can bind to PPARγ, and comprise mainly sulfonyl-indoles.

Dual alpha-gamma agonists are suitable as well, and include glitazars.

Suitable glitazars include aleglitazar, muraglitazar and tesaglitazar.

PPARα agonists include fibrates and biguadines.

Preferably, the compound comprising at least one saccharide and at least one sulphate can be present in the pharmaceutical composition such as in an amount of 0-10 wt. % relative to a total weight.

Preferably, the PPARγ agonist can be present in an amount of 0-10 wt. % relative to a total weight.

Preferably, the relative amounts between the two compounds (sulphated compound to PPARγ agonist) are 1:1 to 1:5.

The pharmaceutically acceptable carrier can be present in an amount of 0.01% to 99.9%, preferably 0.1%-10%, and its amount will depend on the formulation.

In one embodiment, the medicament is in the form of a tablet, suitable for oral administration.

In another embodiment, the medicament is in a form suitable for local administration.

The medicament can comprise the components in the form of a solid, or liquid preparation.

The dosage form can be an immediate release or extended release formulation.

The invention furthermore relates to the pharmaceutical composition of the invention for use in the prevention of metastasis or stopping of ongoing metastasis.

The invention furthermore relates more specifically to the pharmaceutical composition of the invention for the use in the prevention of metastasis or stopping of ongoing metastasis.

It is an advantage of the present invention that the constituents of the composition can be non-biologics, and several of the exemplary components are approved medicaments.

Pentosan polysulphate (PPS), manufactured from beech-wood, is an FDA-approved oral medication for the treatment of interstitial cystitis (IC), also known as painful bladder syndrome. PPS is known to have anti-inflammatory and pro-chondrogenic properties. Pentosan polysulphate is available as pills or as a direct infusion into the bladder.

Adequan, a polysulphated glycosaminoglycan, is a well-known veterinary medicament for treating joint pain.

Indomethacin is an FDA-approved, non-steroidal anti-inflammatory drug (NSAID). It's commonly used to reduce fever, pain, stiffness and swelling. Furthermore, Indomethacin is a COX-inhibitor that blocks prostaglandin production and is used as an inhibitor of inflammation. In higher doses it can also induce adipogenesis in vitro in mesenchymal progenitor cells by activating PPARγ.

Pioglitazone is a powerful PPARγ agonist, and belongs to the class of thiazolidinediones, or glitazones, and was designed to treat type II diabetes by increasing the insulin sensitivity.

### SUMMARY OF THE FIGURES

FIG. 1 shows pictures of histological staining experiments, relating to the synergistic effect of the combination of Pentosan sulphate with Indomethacin.

FIG. 2 shows pictures of histological staining experiments, relating to the synergistic effect of the combination of Pentosan sulphate with Pioglitazone.

FIG. 3 shows pictures of histological staining experiments, relating to the synergistic effect of the combination of Adequan with Indomethacin.

FIG. 4 shows in vitro measurement in relative light units (RLU) of PPAR transcriptional activity in KS-483 cells transfected with a dual PPAR reporter system and exposed to different compounds.

FIGS. 5 shows a schematic structure of Thiazolidinedion and FIG. 6 of sulfonylurea.

FIGS. 7a,b, 8, 9a,b, 10, 11a,b 12, 13a,b, and 14 show experimental results of the claimed composition and synergistic effects thereof, FIGS. 15a-d show stimulation schemes, FIGS. 16a-c and 17a-d staining results, and FIG. 18a,b synergistic effects.

### DETAILED DESCRIPTION OF THE FIGURES

The invention will be elucidated with the following non-limiting examples and experiments.

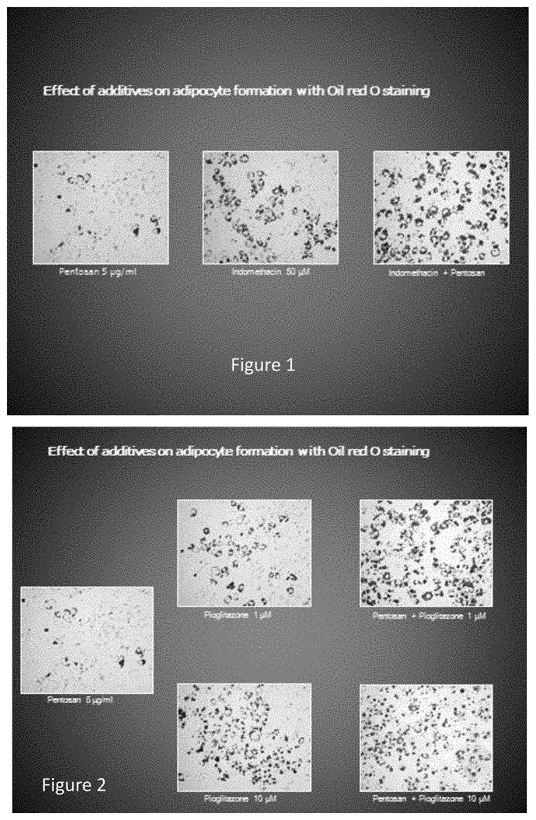

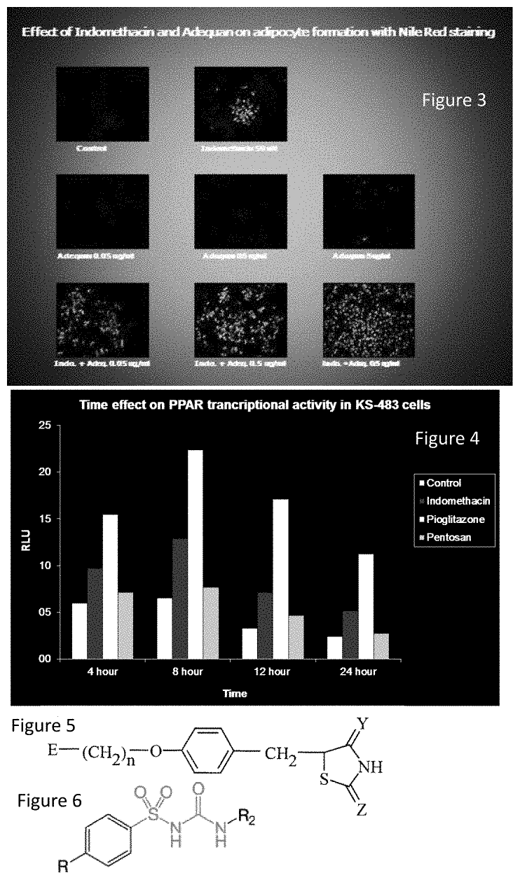

FIG. 1-3 show the effect of the combined use of a compound comprising a carbohydrate containing more than one sulphate (Adequan or Pentosan sulphate), and Indomethacin or Pioglitazone. The concentrations of active compounds used to stimulate the cells are given in the pictures. The active compounds were added to the medium, and thereafter the cells were suspended in the medium. After 3 days, the cells were stained with Oil red O or Nile Red staining.

Oil Red O (Solvent Red 27, Sudan Red 5B, C.I. 26125, C26H24N4O) is a lysochrome (fat-soluble dye) diazo dye used for staining of neutral triglycerides and lipids on frozen sections. The pictures show central nuclei, increasing lipid formation and droplets by the combined use of the composition of the invention, and therefore is indicative of conversion of cancer cells with metastatic potential into harmless cells. Nile red (also known as Nile blue oxazone) is a lipophilic stain accumulating in lipid globules inside the cell. Nile Red fluoresces strongly when partitioned into lipids, but practically not at all in an aqueous solution.

FIG. 4 shows the effect of exposure of KS-483 cells to a compound comprising a carbohydrate and a sulphate (Adequan or Pentosan sulphate), and Indomethacin or Pioglitazone. Similar results are obtained with other cell-lines.

Similar experiments are performed on other cell lines, on organoids, and on assays, showing similar results.

Examples and Experiments

The genes in KS-483 cells were assessed for up- or downregulation after stimulation with the compounds. Briefly, KS-483 cells were cultured under standard culturing conditions in α-MEM medium supplemented with glutamax, penicillin/streptomycin and 10% heat-inactivated Fetal Calf Serum (FCS). Similar experiments were conducted with other cell lines, such as the 3T3-L1 cell line.

From experiments with up- and downregulated genes, and their activity, it is clear that a composition, comprising (i) a compound comprising at least one carbohydrate and containing more than one sulphate, in combination with (ii) indomethacin and/or pioglitazone blocked metastasis development.

Activity Measurements

KS-483 cells were used for activity measurements. From the RLU measured after exposure to single compounds it is clear that a composition, comprising (i) a compound comprising at least one carbohydrate and containing more than one sulphate and indomethacin and/or pioglitazone lead to a significant increase in activity and transfer away from metastatic cells.

The invention has been described by reference to certain embodiments discussed above. It will be recognized that these embodiments are susceptible to various modifications and alternative forms well known to those of skill in the art without departing from the scope of the invention. Accordingly, although specific embodiments have been described, these are examples only and are not limiting upon the scope of the invention, which is defined in the accompanying claims.

Experimental Design Cell Culture

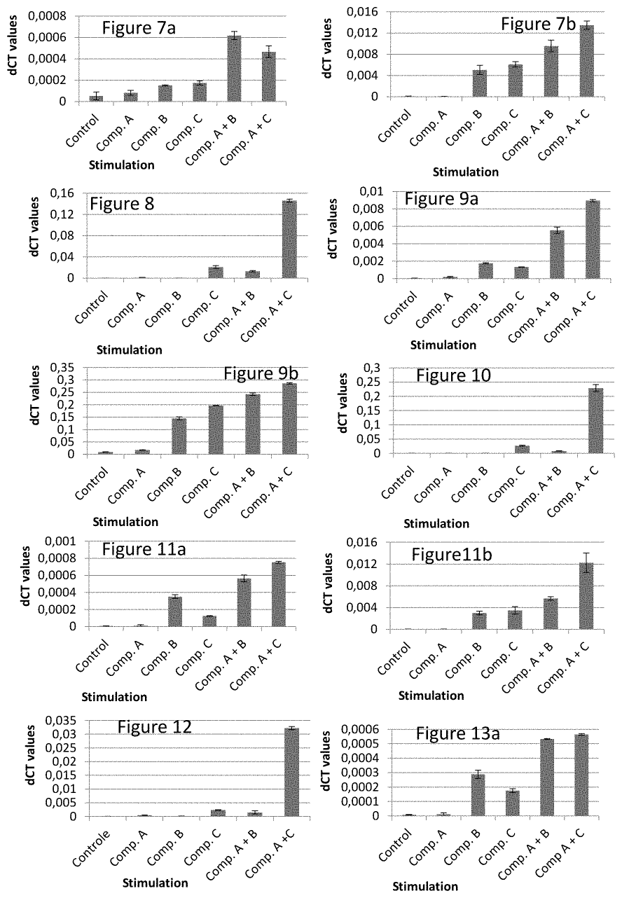

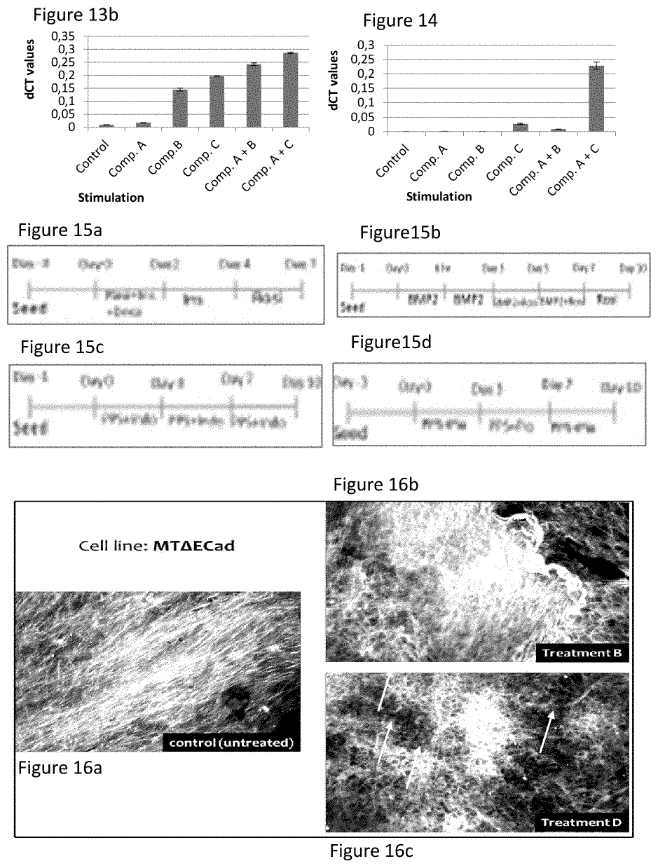

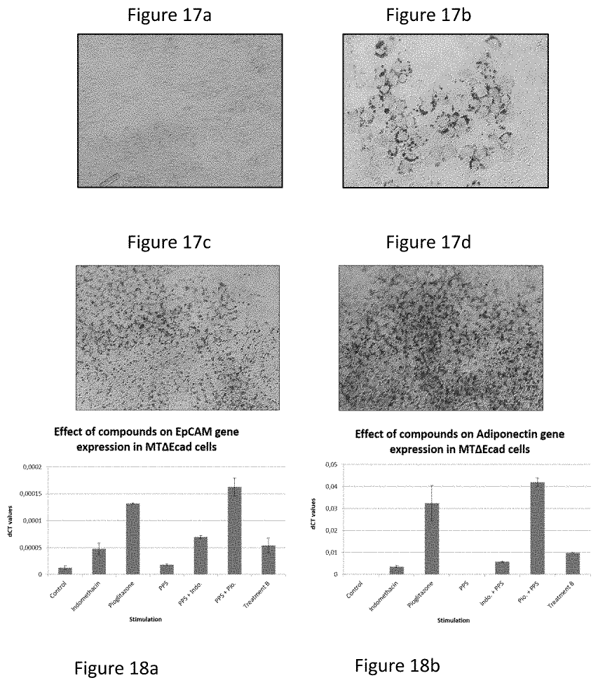

For FIGS. 7a-14 the following conditions were used:

| Protocol 3T3-L1 | Protocol 3T3-L1 | Protocol KS-483 |
|---|---|---|
| cell line αMEM | cell line DMEM | cell line αMEM |
| 10% heat | 10% heat | 10% heat |
| inactivated FCS | inactivated FCS | inactivated FCS |
| Compound A - | Compound A - | Compound A - |
| 5.0 μg/ml | 0.5 μg/ml | 5.0 μg/ml |
| Compound B - | Compound B - | Compound B - |
| 50 μM | 50 μM | 5.0 μM |
| Compound C - | Compound C - | Compound C - |
| 1.0 μM | 1.0 μM | 1.0 μM |
| Compound A + B | Compound A + B | Compound A + B |
| Compound A + C | Compound A + C | Compound A + C |
| | In the of presence of: | |
| | 0.25 μM Dexameth- | |
| | asone & 1 ug/ml Insulin | |

Further Experiments

Background

The overall rationale of the in vitro experiments conducted to test the efficacy of the combined present compounds in the treatment of cancer was mainly based on their capacity to induce terminal adipogenesis and transition into beige/brown fat cells, following experimental conditions of a previous publication with different compounds but similar endpoints (Ishay-Ronen et al., Cancer Cell 2019) https://doi.org/10.1016/j.ccell.2018.12.00

Results.

MTΔECad cells were derived from the MMTV-Neu transgenic mouse, a genetic model of irreversible EMT. MTΔECad cells were treated with the following compounds: Indomethacin (Indo), Pioglitazone (Pio) and Pentosan Polysulphate (PPS)

Treatments & Methods

According to the protocol established by the Ishay-Ronen et al. study, inventors employed a combination of insulin, dexamethasone and Rosiglitazone for 3T3-L1 cells (treatment A), and of Rosiglitazone and BMP2 for MTΔECad cells (treatment B).

For the stimulation of MTΔECad cells the following concentrations were used: PPS (5 μg/ml), Indomethacin (50 μM), en Pioglitazone (10 μM).

Untreated cell lines were employed as negative controls. As read-outs of adipogenesis, several approaches were tested:
1. Oil red O staining by histochemistry.
2. Nile Red staining by immuno-fluorescence (IF)
3. RTqPCR (gene expression analysis)
Adiponectin (adipocyte marker)
EpCam (marker for epithelial cells) FIGS. 15a-d show stimulation schemes; FIGS. 15a-b according to prior art, and FIGS. 15c-15d according to the present invention.

The FIG. 16 shows Nile Red staining of the MTΔECad treated cells. Induction of adipogenesis (indicated by the red staining), epithelialization (i.e. re-acquisition of epithelial features, i.e. MET; indicated by morphological changes from spindle-shaped to cuboid cells) and induction of adipogenesis is shown in both conditions:

Control=untreated MTΔECad cells.

Treatment B (FIG. 15ba)=Rosiglitazone and BMP2 (according to Ishay-Ronen et al., Cancer Cell 2019)

Treatment C and D (FIG. 15c-d)=PPS and indomethacin and PPS en Pioglitazone (the present combinations).

Although the images below are only indicative of the processes triggered by the different treatments and do not allow their quantitative evaluation, they point to the fact that the two processes, that is adipogenesis and transition into fat cells can occur independently of each other. Interestingly, this is thought to prevent and even ‘cure’ metastasis formation.

FIG. 16. Left Control (FIG. 16a) untreated MTΔECad cells that clearly show spindle shaped cells characteristics for epithelial cells that have undergone EMT. On the right (FIG. 16b) MTΔECad cells of which many are differentiated into adipocytes as a positive control (red stained) by treatment B (FIG. 16b) and by treatment D (FIG. 16c). Important to note is that the MTΔECad cells that did not differentiate into adipocyte by treatment B or D have a more cuboidal phenotype characteristic of epithelial cells in contrast to the spindle shape seen in the untreated control cells. Arrows indicate abundantly present fat cells.

FIG. 17 shows Oil Red O staining of the MTΔECad treated cells. Under these culture conditions, adipocyte formation has been found after stimulation with the combination of Indomethacin/PPS and Pioglitazone/PPS stimulated cells. Pioglitazone alone can stimulate adipocyte formation at a low level. FIG. 17a: control; FIG. 17b indomethacine+pentosan; FIG. 17c pioglitazone; FIG. 17d: pioglitazone+pentosan.

EpCAM Gene Expression.

Inventors studied the effect of treatment of MTΔECad cells with the different compounds and combinations on EpCAM expression as a marker for epithelial cells and Adiponectin expression as a marker for adipocytes.

qPCR

Steps performed when measuring gene expression using real-time PCR.

RNA was first isolated and characterized for quantity and integrity. During a two-step reaction, cDNA was first synthesized and then used as a PCR template. The housekeeping gene GAPDH was used for normalizing the results. A normalization factor has been used for calculating each individual sample. Dividing the fluorescent data by its normalization factor produced the normalized data, which was followed by statistical analysis.

Results

EpCam

Indomethacin and Pioglitazone clearly stimulated EpCAM expression. PPS on its own had no significant effect. However when PPS was combined with Indomethacin or Pioglitazone there was a synergistic effect. 3T3-L1 pre-adipogenic cells unstimulated or stimulated towards adipocytes (treatment A) were used as a control because they are mesenchymal cells and therefore do not express EpCAM, data not shown. Cells treated as a positive control (treatment B) show stimulated EpCAM gene expression.

MTΔECad treated cells show synergetic upregulation of EpCam gene expression after stimulation with the combination Indomethacin/PPS or Pioglitazone/PPS, This indicates that MTΔECad treated cells express more epithelial characteristics, since EpCam is only expressed on epithelial cells and not on mesenchymal cells.

Adiponectin

MTΔECad treated cells shown synergetic upregulation of adiponectin gene expression after stimulation with the combination Indomethacin/PPS or Pioglitazone/PPS, indicating that the treated cells express adipocyte characteristics, since adiponectin expression is restricted to adipocytes.

3T3-L1 pre-adipogenic cells were stimulated towards adipocytes (treatment A) and used as a control, Adiponectin expression was up-regulated after stimulation, data not shown.

Indomethacin and Pioglitazone obviously stimulated adiponectin gene expression and a clearly synergetic effect is shown after stimulation with PPS in combination with Indomethacin or Pioglitazone. PPS on its own had no effect on adiponectin gene expression.

Cells treated as a positive control (treatment B) shown stimulated adiponectin gene expression. See FIGS. 18a,b. Conclusion

Both Indomethacin and Pioglitazone in combination with Pentosan Polysulfate (PPS) are able to terminally differentiate MTΔECad cells breast cancer cells into adipocytes but at the same time they can induce MET (mesenchymal-to-epithelial transition). This was shown by appearance in culture of Nile Red positive lipid droplets containing adipocyte like cells that also express Adiponectin, measured by qPCR. However, at the same time cuboidal non-adipocyte cells are visible indicative of epithelial cells which was confirmed by the fact that we also found expression of the epithelial marker EpCAM using qPCR.

FIGS. 7a,b, 8, 9a,b, 10, 11a,b, 12, 13a,b and 14 show experimental results of the claimed composition and synergistic effects thereof. FIGS. 7a,b show the effect of compounds on Perilipin-1 gene expression in 3T3-L1 cells; in FIG. 7a the effect of the compounds alone, and in FIG. 7b the effect of compounds stimulated in presence of Insulin/Dexamethasone. FIG. 8 shows the effect of compounds on Perilipin-1 gene expression in KS-483 cells. FIGS. 9a,b show the effect of compounds on FABP4 gene expression in 3T3-L1 cells; in FIG. 9a the effect of the compounds alone, and in FIG. 9b the effect of compounds stimulated in presence of Insulin/Dexamethasone. FIG. 10 shows the effect of compounds on FABP4 gene expression in KS-483 cells. FIGS. 11a,b show the effect of compounds on CD-36 gene expression in 3T3-L1 cells; in FIG. 11a the effect of the compounds alone, and in FIG. 11b the effect of compounds stimulated in presence of Insulin/Dexamethasone. FIG. 12 shows the effect of compounds on CD-36 gene expression in KS-483 cells. FIGS. 13a,b show the effect of compounds on Adiponectin gene expression in 3T3-L1 cells; in FIG. 13a the effect of the compounds alone, and in FIG. 13b the effect of compounds stimulated in presence of Insulin/Dexamethasone. FIG. 14 shows the effect of compounds on Adiponectin gene expression in KS-483 cells.

1. A pharmaceutical composition for a treatment selected from the prevention of metastasis and stopping of ongoing metastasis, comprising (i) a first compound comprising at least one carbohydrate and containing more than one sulphate, in combination with (ii) a second compound selected from the group of thiazolidinediones, NSAIDs, sulphonylureas, and indoles.

2. The pharmaceutical composition for a treatment selected from the prevention of metastasis and stopping of ongoing metastasis according to claim 1, comprising at least one of pentosan sulphate, and adequan.

3. The pharmaceutical composition for a treatment selected from the prevention of metastasis and stopping of ongoing metastasis according to claim 1 2, comprising at least one of indomethacin and pioglitazone.

4. The pharmaceutical composition a treatment selected from in the prevention of metastasis and stopping of ongoing metastasis according to claim 1, wherein the first compound and second compound are provided in a molar ratio of 0.01:1 to 1:0.01.

5. The pharmaceutical composition a treatment selected from in the prevention of metastasis and stopping of ongoing metastasis according to claim 1, wherein the composition further comprises (iii) at least one pharmaceutically acceptable carrier.

6. The pharmaceutical composition a treatment selected from in the prevention of metastasis and stopping of ongoing metastasis according to claim 1, wherein the active pharmaceutical ingredients are in one dosage form.

7. The pharmaceutical composition a treatment selected from in the prevention of metastasis and stopping of ongoing metastasis according to claim 1, comprising at least one of pentosan sulphate and adequan, and at least one of indomethacin and pioglitazone.

8. The pharmaceutical composition a treatment selected from in the prevention of metastasis and stopping of ongoing metastasis according to claim 1, wherein the pharmaceutical composition comprises separate dosage forms for individual pharmaceutical active ingredients, and wherein the composition is in the form of at least one of a tablet, a capsule, a repository, and an injectable.

9. The pharmaceutical composition for a treatment selected from the prevention of metastasis and stopping of ongoing metastasis according to claim 8, wherein the composition is in the form of at least one of a tablet and a capsule suitable for oral administration.

10. The pharmaceutical composition for a treatment selected from the prevention of metastasis and stopping of ongoing metastasis according to claim 1, formulated in a form, which, depending on its use, allows targeting of at least one of specific cells, tissues, and organs.

11. The pharmaceutical composition for a treatment selected from the prevention of metastasis and stopping of ongoing metastasis according to claim 1, wherein the cancer originates from epithelial tissue.

12. The pharmaceutical composition for a treatment selected from the prevention of metastasis and stopping of ongoing metastasis according to claim 11, wherein the cancer is selected from skin cancer, lung cancer, stomach cancer, brain cancer, breast cancer, kidney cancer, liver cancer, colo-rectal cancer, bladder cancer, gall bladder cancer, prostate cancer, pancreas cancer, head/neck cancer, melanomas, and esophagus cancer.

13. The pharmaceutical composition for a treatment selected from the prevention of metastasis and stopping of ongoing metastasis according to claim 1 for use as a medicament by administering said medicament in an effective amount to cancer cells with metastatic potential for a sufficient period.

14. The pharmaceutical composition for a treatment selected from the prevention of metastasis and stopping of ongoing metastasis of claim 13, wherein the administration is to a pet or mammal comprising said tissue, wherein the mammal preferably is a human with a cancer that has metastatic potential.

15. A medicament comprising a pharmaceutical composition comprising (i) a first compound comprising at least one carbohydrate and containing more than one sulphate, in combination with (ii) a second compound selected from the group of thiazolidinediones, NSAIDs, sulphonylureas, and indoles in a treatment selected from for prevention of metastasis and for stopping of ongoing metastasis.

16. The pharmaceutical composition according to claim 1, wherein the first compound comprising at least one carbohydrate and containing more than one sulphate is selected from multiple Sulphur containing agents with a molecular weight of <10 kDa.

17. The pharmaceutical composition according to claim 6, wherein the dosage form comprises 1-10 mg active ingredients/kg body weight.
